SEQUENCE LISTING (10 sequences)

SEQ ID NO: 1 - protein, 34 aa, Artificial sequence
  Fibrinogen  chain (P02675)
AHYGGFTVQNEANKGGETSEMYLIQPDSSVKPYR

SEQ ID NO: 2 - protein, 15 aa, Artificial sequence
  Colligin 2 (P50454)
LYGPSSVSFADDFVR

SEQ ID NO: 3 - protein, 10 aa, Artificial sequence
  Acidic calponin (Q15417)
YDHQAEEDLR

SEQ ID NO: 4 - protein, 417 aa, Artificial sequence
  marker protein colligin 2
MRSLLLGTLCLLAVALAAEVKKPVEAAAPGTAEKLSSKATTLAEPSTGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFPDKRSALQSINEWAAQTTDGKLPEVTKDVERTDGALLVNAMFFKPHWDEKFHHKMVDNRGFMVTRSYTVGVTMMHRTGLYNYYDDEKEKLQLVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRLKGDKMRDEL

SEQ ID NO: 5 - protein, 418 aa, Artificial sequence [sequence omitted]

SEQ ID NO: 6 - protein, 2355 aa, Artificial sequence
  marker protein fibronectin
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 7 - protein, 445 aa, Artificial sequence [sequence omitted]

SEQ ID NO: 8 - protein, 512 aa, Artificial sequence [sequence omitted]

SEQ ID NO: 9 - protein, 391 aa, Artificial sequence
  marker protein fibrinogen
MKHLLLLLLCVFLVKSQGVNDNEEGFFSARGHRPLDKKREEAPSLRPAPPPISGGGYRARPAKAAATQKKVERKAPDAGGCLHAETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 10 - protein, 329 aa, Artificial sequence
  marker protein acidic calponin 3
MTHFNKGPSYGLSAEVKNKIASKYDHQAEEDLRNWIEEVTGMSIGPNFQLGLKDGIILCELINKLQPGSVKKVNESSLNWPQLENIGNFIKAIQAYGMKPHDIFEANDLFENGNMTQVQTTLVALAGLAKTKGFHTTIDIGVKYAEKQTRRFDEGKLKAGQSVIGLQMGTNKCASQAGMTAYGTRRHLYDPKMQTDKPFDQTTISLQMGTNKGASQAGMLAPGTRRDIYDQKLTLQPVDNSTISLQMGTNKVASQKGMSVYGLGRQVYDPKYCAAPTEPVIHNGSQGTGTNGSEISDSDYQAEYPDEYHGEYQDDYPRDYQYSDQGIDY

(19) United States
(12) Patent Application Publication
(10) Pub. No.: US 2010/0240088 A1
(43) Pub. Date: Sep. 23, 2010

(54) Peptide Markers for Diagnosis of Angiogenesis

(75) Inventors: Theo Marten Luider, Rotterdam (NL); Petrus Abraham Elisa Sillevis Smitt, Rotterdam (NL)

Correspondence Address: SWANSON & BRATSCHUN, L.L.C., 8210 SOUTHPARK TERRACE, LITTLETON, CO, 80120, US

(73) Assignee: ERASMUS UNIVERSITY MEDICAL CENTER ROTTERDAM, Rotterdam (NL)

(21) Appl. No.: 12/522,880

(22) PCT Filed: Jan. 14, 2008
(86) PCT No.: PCT/NL08/00019

§ 371 Date: Nov. 24, 2009

(30) Foreign Application Priority Data
Jan. 12, 2007 (NL) NL2007/050014

(51) Int. Cl. C12Q 1/02; G01N 33/68; C07K 2/00; C07K 14/78

(52) U.S. Cl. 435/29; 436/86; 530/300; 530/356

(57) ABSTRACT

The present invention relates to a method for detecting physiological or pathological blood vessel formation, preferably glioma activity, comprising determining the expression level of colligin 2 in blood, cerebrospinal fluid or tissue vasculature. The invention further relates to the use of a method for detecting physiological or pathological blood vessel formation wherein said use is for monitoring a disease process; a healing process; or a response to a disease therapy.

### FIELD OF THE INVENTION

The present invention is in the field of disease diagnostics. In particular, the invention relates to the detection of peptides and/or proteins as markers for the diagnosis, prognosis, or therapeutic monitoring of angiogenesis and physiological or pathological processes characterized by angiogenesis such as tumorigenesis, ischemia and/or wound healing. The invention further relates to markers and to methods for detection of diseases such as cancer, in particular glioma. The invention further provides the use of colligin 2 as a marker for the diagnosis, prognosis, or (therapeutic) monitoring of angiogenesis and physiological or pathological processes characterized by angiogenesis such as tumorigenesis, ischemia and/or wound healing.

### BACKGROUND OF THE INVENTION

Gliomas are the most common primary brain tumors. The diagnosis of these tumors and the decisions regarding therapy is based almost exclusively on the tissue histopathology. Diffuse gliomas are highly infiltrative and heterogeneous. Gliomas are among neoplasms with highest degree of vascularisation. The growth of gliomas largely depends on their blood supply. The elimination of the blood supply would result in the destruction of these tumors. Despite the elucidation of many genetic aberrations of gliomas over the last decades, only few useful biomarkers or therapeutic targets have been identified so far. Despite the gradual unraveling of the roles of a large number of regulatory proteins in the process of tumor neovascularisation, no major steps forward in antiangiogenic therapies for gliomas have been recorded to date. The identification of more tumor vasculature-related proteins may result in the finding of new targets of anti-angiogenic therapies and understanding of the formation of neovasculature in glioma.

Rapid and major developments in proteomic technology and methodology over the last decade have opened a new stage in the identification of proteins. Matrix-assisted laser desorption/ionization time-of-flight mass spectrometry (MALDI-TOFMS) recently became available as a flexible tool in the search for disease markers. Moreover, the recently introduced technique of matrix-assisted laser desorption\ionization Fourier transform mass spectrometry (MALDI-FTMS) provides a powerful technique for accurate peptides mass measurements. This technique has successfully been used for studies in protein interactions and post-translational modifications of proteins.

Hitherto only low-molecular weight caldesmon was suggested as a potential marker for glioma (Zheng et al., Clin Cancer Res. 2005, 11:4388-4392). However, there is a need for additional biomarkers or therapeutic targets, in particular protein or peptide markers that can indicate the presence of angiogenesis and physiological or pathological processes characterized by angiogenesis such as tumorigenesis, ischemia and/or wound healing.

It is an aim of the present invention to provide novel markers for medical and veterinary diagnosis of angiogenesis and physiological or pathological processes characterized by angiogenesis such as tumorigenesis, ischemia and/or wound healing.

### SUMMARY OF THE INVENTION

By using a combination of different MALDI-MS techniques for marker detection, the present inventors have now found that colligin 2, a collagen-binding protein involved in collagen biosynthesis, localized to the endoplasmic reticulum and belonging to the superfamily of serine protease inhibitors, is a powerful biomarker for blood vessel formation in general and for angiogenesis and/or vasculogenesis of tumors in particular.

In addition to colligin 2, also identified and disclosed herein as markers indicative for blood vessel formation in general and for angiogenesis and/or vasculogenesis of tumors in particular fibronectin, fibrinogen and acidic calponin 3, and these markers may be used instead of colligin 2 in exactly the same manner in all aspects of the invention as disclosed herein.

In a first aspect, the present invention provides a method for detecting physiological or pathological blood vessel formation, comprising determining the expression level of colligin 2 in blood, cerebrospinal fluid or tissue vasculature.

In a preferred embodiment of said method, said physiological or pathological blood vessel formation is indicative of tumor activity, preferably glioma activity; ischemia; and/or wound healing.

In another preferred embodiment of said method, said tissue is a tumor, preferably a glioma.

In a preferred embodiment of said method, said expression level is determined by detecting the colligin 2 protein, or a peptide fragment thereof in a mass range of 800 to 27,000 Da. Also, a transcription product of the colligin 2 gene, such as an mRNA from the colligin 2 gene may be determined in order to determine the expression level of colligin 2.

In methods of the present invention, the peptides are suitably detected by MALDI MS analysis and therefore will generally be digested, for instance by trypsin, and detected when having a molecular mass in a range of 400-20,000, preferably in a range of 800 to 4,000 Da. The nucleic acids, such as the mRNAs transcribed from the colligin 2 gene may be detected for instance with RT-PCR (reverse-transcriptase polymerase chain reaction) optionally in combination with a suitable method for detecting DNA amplification products produced in such a reaction.

In yet a further embodiment of said method, in addition to said expression level of colligin 2, also the expression level of one or more of the proteins selected from fibronectin, fibrinogen, and acidic calponin 3 is determined.

In a preferred embodiment of said method, said detection is performed by immunohistochemistry or mass spectrometry.

In another aspect, the present invention provides the use of a method for detecting physiological or pathological blood vessel formation as described above, wherein said use is for monitoring a disease process; a healing process; or a response to a disease therapy. Therapeutic monitoring of disease means monitoring of disease activity and treatment response or responsiveness.

In a preferred embodiment of said use, said disease is cancer or ischemia; or wherein said healing process is a wound healing process or tissue repair process following ischemia; or wherein said disease therapy is anti-tumor therapy.

In another aspect, the present invention provides a marker protein or marker peptide for detecting physiological or pathological blood vessel formation, wherein said marker protein is colligin 2 and said marker peptide is a peptide fragment of colligin 2 having a mass of between 800 and 27,000 Da.

In a preferred embodiment of said marker protein or peptide, said physiological or pathological blood vessel formation is related to vasculogenesis, preferably vasculogenesis in tumorigenesis, preferably glioma activity; ischemia; or wound healing.

In another aspect, the present invention provides a marker profile for detecting physiological or pathological blood vessel formation, preferably glioma activity, in a subject wherein said marker profile comprises the expression level in blood, cerebrospinal fluid or tissue vasculature of a subject of a first protein being colligin 2 or a peptide thereof, and wherein said marker profile further comprises at least one additional expression level of a protein or peptide fragment selected from the group of fibronectin, fibrinogen and acidic calponin 3.

In yet another aspect, the present invention provides for the use of a marker or marker profile of the invention for the detection of physiological or pathological blood vessel formation, preferably in relation to trauma, ischemia or surgery, or glioma.

It is known in the art of proteome analysis that factors such as sample stability and a low number of measurements per sample can cause difficulties regarding the reproducibility of proteomic profiling studies. Also, it is known that there is low reproducibility of peak height in MALDI-TOF MS. The method of the present invention overcomes these problems in several ways and is less affected by these variations. First, the samples are all handled in a standardized way. Secondly, the sample preparation method is uncomplicated and straightforward. Thirdly, the height of the peaks is not included in the analysis because quantitative measurements of peak heights with MALDI TOF MS are poorly reproducible, with standard deviations up to 30%. In the present method only the absence or presence of the peaks is scored. (see the Examples below for details)

Preferred embodiments of the method of the present invention include for instance the detection of the marker protein or marker peptide in a sample of body tissue, tumor tissue, CSF or blood (or serum) of a subject by MALDI-FT mass spectrometry, (MALDI) Triple-quad mass spectrometry or an immunoassay, such as ELISA or immunohistochemistry. The tissue or fluid sample is prepared for such analyses by methods well known to the skilled person.

Samples used in aspects of the present invention may be obtained by biopsy or puncture, involving the removal of a small portion of tissue from the body, such as needle biopsy or open biopsy. Alternatively, the sample may be body liquids such as blood, serum, liquor, cerebrospinal fluid or the like.

Samples used in aspects of the present invention may be unprocessed, or processed samples, meaning that the samples may or may not have been subjected to procedures wherein the biological, physical or chemical composition of the sample is altered. The samples may also be subjected to multiple processing steps. Highly preferred samples are samples of blood vessels. Most preferred samples in the case of a tumor are samples of blood vessels of said tumor.

In an alternative embodiment of a method of the invention, the optionally processed samples are body tissue samples processed by subjecting said samples to laser capture microdissection to provide collections of microdissected cells, said collections preferably amounting to about 200-3,000 cells. Preferably, said collections of microdissected cells are provided in the form of pooled collections of microdissected cells.

In yet another alternative embodiment of a method of the invention the optionally processed samples are body tissue samples, body fluid samples, or collections of microdissected cells, optionally processed by subjection to protein digestion, preferably using trypsin, to provide optionally processed samples comprising proteins or peptide fragments from the proteins in said samples. Thus, the method optionally comprises the step of cleaving the proteins in a sample (i.e. polypeptides in general) with a (optionally sequence specific) cleavage agent to form peptide fragments, optionally followed by deactivating the cleavage agent. A sequence specific cleavage agent in aspects of the present invention preferably cleaves the polypeptides on the C-terminal side of a lysine residue. The specific cleavage agent preferably comprises Lys-C or Trypsin. The cleavage agent is preferably trypsin. Polypeptide cleaving (e.g. trypsin digestion) is performed to provide peptide fragments sufficiently small to be analysed by MALDI analysis. However, some samples may comprise peptide fragments of sufficiently small size to allow direct MALDI analysis. Examples of peptides that can be detected or analyses in unprocessed samples include (neuro)peptides, hormones, etc.

In principle, any body tissue of a subject may be used in aspects of the invention. Suitably a body tissue is selected from the group consisting tissues of brain, lung, heart, prostate, esophagus, stomach, jejunum, ileum, caecum, colon, gall bladder, bile duct, breast, ovary, testicle, lymph node, thymus, kidney, liver, muscle, nerve, bone, bone marrow, and placenta. A highly preferred tissue sample is a blood vessel sample, even more preferably a blood vessel of the brain.

The body fluid analysed in a method of the present invention may suitably be selected from the group, consisting of blood, serum, cerebrospinal fluid (CSF), urine, saliva and semen. Highly preferred fluid samples are blood, serum, and cerebrospinal fluid (CSF).

Body fluid samples, when used in methods of the invention, may suitably be provided in sample volumes of between 0.01 and 100 μl. However, it is a particular advantage of the present invention that very small sample volumes will generally suffice. An amount in a range from 0.1-25 μl, preferably in a range from 1-10 μl of optionally processed body fluid is generally sufficient for MALDI-FT-ICR mass spectrometric analysis. A suitable sample fluid preferably comprises about 0.05-5 mg/ml of protein.

Herein below, the terms “patient” and “subject” are used interchangeably to indicate animal subjects, including human and non-human subjects that are in need of disease diagnosis.

In yet another aspect, the present invention provides a method for detecting glioma, comprising measuring the expression level of a marker protein selected from the group consisting of fibronectin, fibrinogen, colligin 2 and acidic calponin 3, preferably colligin 2, in blood, CSF or glioma vasculature samples of patients.

In another aspect, the present invention provides a method for monitoring disease activity of glioma and/or the response to a treatment regimen, comprising measuring the expression level of fibronectin, fibrinogen, colligin 2 and/or acidic calponin 3 in blood, CSF and/or glioma vasculature samples of patients.

In the various methods described in the present invention the step of detecting the marker peptide or marker protein in a sample may suitably be performed by 1VIALDI Triple-quad analysis of proteins and peptides in a tissue sample to quantify said marker protein or marker peptide indicative for a specific disease in suspect diseased tissue samples of subjects.

### SHORT DESCRIPTION OF THE DRAWINGS

FIG. 1: hypertrophied vessels in high-grade glioma. The counter stain of a glioma section shows the hypertrophied vessels in the sample (arrows). These vessels were our target to be microdissected.

FIG. 2: heat map of unsupervised clustering of the following four groups: Group no. 1, glioma blood vessels, group no. 2, normal brain blood vessels, group no. 3, glioma surrounding tissue, group no. 4, normal brain surrounding tissue. The figure illustrates a close up of an unsupervised clustering dendrogram based on peptide masses and group of samples on spotfire. The cluster masses are displayed on the x-axis, whereas the y-axis represents the samples ordered by group. Red blocks show the presence of peptide in the spectrum of the sample. The unsupervised clustering of the samples results in clustering of eight out of ten glioma blood vessel samples, group no. 1. One of the two samples that did not cluster had a poor spectrum, this one clustered with the other poor spectrum sample of normal surround tissue at the top of the heat map. The other glioma sample did not cluster. While clustering based on peptide masses showed a specific peptide pattern of glioma blood vessels group. Those peptides appeared exclusively in glioma blood vessels group.

FIG. 3: immunohistochemistry for fibronectin in glioma and normal brain samples. A: the strong positive staining of fibronectin protein in the hypertrophied vessels of glioma sample. B: the negative staining of fibronectin protein in normal brain vessels. C: some of the normal brain vessels showed a very faint staining for fibronectin.

FIG. 4: immunohistochemistry for colligin 2 protein in glioma and normal brain samples. A: the strong positive staining of colligin 2 protein in the hypertrophied vessels of glioma sample. B: the negative staining of colligin 2 protein in normal brain vessels.

FIG. 5. Results of immunostaining of various tissue samples for colligin 2 and fibronectin. A, anaplastic oligodendroglioma; B, ependymoma; C, renal cell carcinoma; D, arteriovenous malformation in brain; E, cavernous angioma; F, contusio cerebri; G, inflammation of skin; H, placenta; I, endometrium. Staining patterns for both colligin 2 and fibronectin are confined to blood vessels. In the case of active blood vessel formation in tumors and in reactive and normal tissues, staining is present. The AVM (D) and the cavernous hemangioma (E) remained largely immunonegative for colligin 2. However, at a single site of recanalization of a thrombosed vessel in the AVM (arrow), positive staining is present. H&E, hematoxylin and eosin

FIG. 6-9 provide the amino acid sequences of the marker proteins colligin 2 (FIGS. 6a and 6b), fibronectin (FIG. 7), fibrinogen (FIG. 8a-c), and acidic calponin 3 (FIG. 9).

### DETAILED DESCRIPTION OF THE PREFERRED EMBODIMENTS

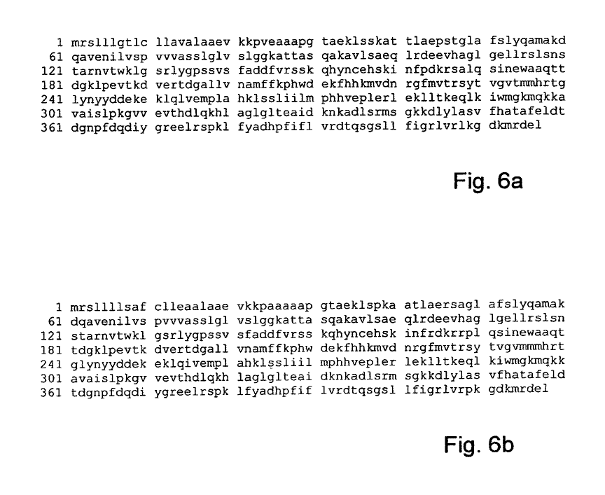

The term “colligin” as used herein, refers to the 47 kD cell surface associated glycoprotein that binds to both gelatin and collagen, also known as CBP2; HSP47; collagen-binding heat-shock protein GP46; colligin 2; myoblast GP46 having the sequence as provided in SEQ ID NO:1 and FIGS. 6a and 6b herein, which Figures present two different isoforms of the protein. It should be stressed that the invention covers also other isoforms of this protein. The skilled person will understand that deviations and mutation may occur within the amino acid sequence or gene sequence of colligin 2, which deviations and mutations are encompassed in the term colligin 2 as used herein.

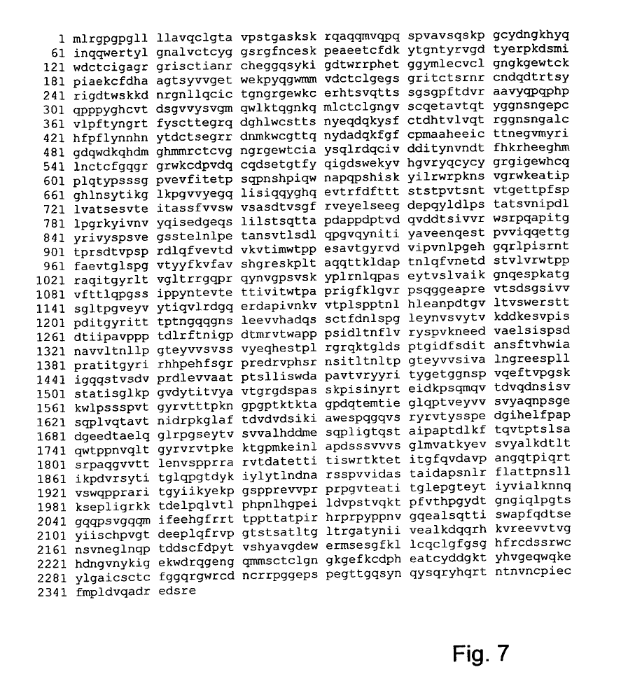

The term “fibronectin” as used herein refers to the protein essentially having the amino acid sequence as shown in FIG. 7. When reference is made to the marker, peptide fragments of the fibronectin protein can also be detected and serve as markers, such as peptides of the protein obtained by enzymatic (tryptic) digestion of samples of a subject wherein said marker is to be detected. In particular, suitable peptide fragments are fragments of fibronectin having a length of 7-100 amino acids, preferably 10-30 amino acids.

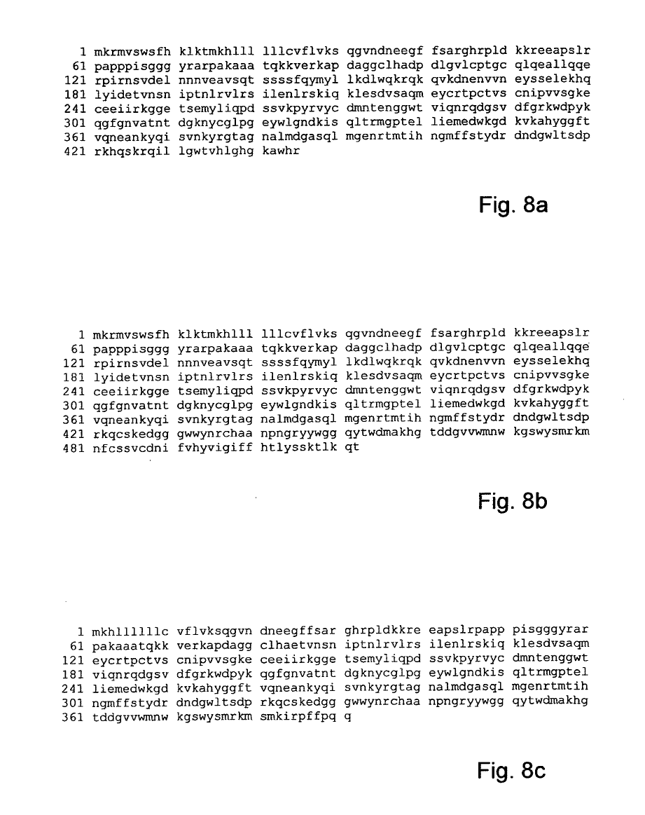

The term “fibrinogen” as used herein refers to the protein fibrinogen beta chain essentially having the amino acid sequence as shown in FIGS. 8a, 8b and/or 8c, which Figures present three different isoforms (isoform CRA_g, isoform CRA_i, and isoform CRA_f, respectively) of the protein. It should be stressed that the invention covers also other isoforms of this protein. When reference is made to the marker, peptide fragments of the fibrinogen protein can also be detected and serve as markers, such as peptides of the protein obtained by enzymatic (tryptic) digestion of samples of a subject wherein said marker is to be detected. In particular, suitable peptide fragments are fragments of fibrinogen having a length of 7-100 amino acids, preferably 10-30 amino acids.

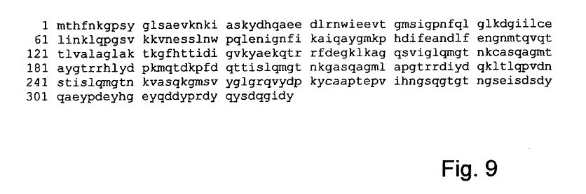

The term “acidic calponin 3” as used herein refers to the protein essentially having the amino acid sequence as shown in FIG. 9. When reference is made to the marker, peptide fragments of the acidic calponin 3 protein can also be detected and serve as markers, such as peptides of the protein obtained by enzymatic (tryptic) digestion of samples of a subject wherein said marker is to be detected. In particular, suitable peptide fragments are fragments of acidic calponin 3 having a length of 7-100 amino acids, preferably 10-30 amino acids.

The term “physiological or pathological blood vessel formation” as used herein, refers to the process of angiogenesis and vasculogenesis, as it occurs as in relation to wound healing and healing of damaged tissues, such as caused by trauma, ischemia, or surgery, or as it occurs as in relation to cancer in tumors. Preferably the term relates to vasculogenesis.

The term “tumor” as used herein, refers to all neoplastic cell growth and proliferation, whether malignant or benign, and all pre-cancerous and cancerous cells and tissues. Preferably the term does not include reference to squamous cell carcinomas.

The terms “cancer” and “cancerous” refer to or describe the physiological condition in mammals that is typically characterized by unregulated cell growth and angiogenesis. New blood vessel development is an important process in tumor progression. It favors the transition from hyperplasia to neoplasia i.e. the passage from a state of cellular multiplication to a state of uncontrolled proliferation characteristic of tumor cells. Examples of cancer include but are not limited to, pancreatic cancer, prostate cancer, breast cancer, colorectal cancer, gastrointestinal cancer, colon cancer, lung cancer, hepatocellular cancer, cervical cancer, ovarian cancer, liver cancer, bladder cancer, cancer of the urinary tract, thyroid cancer, renal cancer, carcinoma, melanoma, and brain cancer.

The term “glioma” as used herein refers to a tumor that arises from glial cells, and which can be encountered in the brain, the spinal cord or any other part of the central nervous system (CNS), such as the optic nerve.

The term “ischemia”, as used herein, refers to an absolute or relative shortage of the blood supply or an inadequate flow of blood to an organ, body part or tissue. Relative shortage refers to the discrepancy between blood supply (oxygen delivery) and blood request (oxygen consumption by tissue). The restriction in blood supply, generally due to factors in the blood vessels, is most often, but not exclusively, caused by constriction or blockage of the blood vessels by thromboembolism (blood clots) or atherosclerosis (lipid-laden plaques obstructing the lumen of arteries). Ischemia results in damage or dysfunction of tissue. Ischemia of the heart muscle results in angina pectoris. Ischemia as referred to herein includes, but is not limited to stroke/transient ischemic attack or cerebrovascular attack, myocardial infarction, myocardial ischemia or ischemic heart disease (angina pectoris), any cardiomyopathy complicated by myocardial ischemia (for instance symptomatic aortic stenosis, HOCM), cerebral bleeding, peripheral (unstable) angina pectoris, claudicatio intermittens (peripheral atherosclerotic artery disease) and other major abnormalities occurring in the blood vessels such as coronary and cerebrovascular diseases as well as to peripheral vascular diseases.

As used herein, the term “angiogenesis” refers to the growth of blood vessels in tissue, and particularly involving the growth of new blood vessels from pre-existing vessels.

As used herein, the term “vasculogenesis” (also referred herein as “neovascularisation” or “neoangiogenesis”) is the formation of blood vessels when there are no pre-existing blood vessels, i.e. the new growth of blood vessels in tissue.

As used herein, the term “tissue” refers to any tissue in which angiogenesis or neoangiogenesis may be detected.

As used herein, the term “wound healing” refers to the healing of any opening in the skin, mucosa or epithelial linings, including openings generally being associated with exposed, raw or abraded tissue, and including, but not limited to first, second and third degree burns, surgical incisions, including those of cosmetic surgery; wounds, including lacerations, incisions, and penetrations; and ulcers including decubital ulcers (bed-sores) and ulcers or wounds associated with diabetic, dental, haemophilic, malignant and obese patients.

The diseases for which diagnosis, prognosis or therapeutic monitoring can be provided through provision of the marker provided herein are in particular tumors such as glioma, and ischemia. Any physiological or pathological process characterized by angiogenesis in a patient can be diagnosed, prognosed or monitored by the markers of the present method. A patient can be any animal, but is preferably a human patient.

Markers for Detecting Physiological or Pathological Processes Characterized by Angiogenesis in a Patient

The present inventors set out to identify proteins that are specifically expressed in glioma vasculature, but not in the normal blood vessels of the brain. The present inventors identified several proteins that were specifically expressed in glioma vasculature by using a method comprising the following steps:

(a) providing an optionally processed (e.g. trypsin digested) sample of a diseased body tissue or fluid as a test sample (i.e. a glioma), and an optionally processed sample of a corresponding healthy body tissue or fluid as a reference sample, wherein said samples comprise peptides and/or proteins;

(b) subjecting both test and reference sample to MALDI-FT-ICR mass spectrometry to generate mass spectra for individual peptides in each sample and to quantify the amount of individual peptides present in each sample;

(c) comparing the amount of an individual peptide present in the test sample with the amount of a peptide having a corresponding mass in the reference sample to generate a list of peptides differentially expressed between test and reference sample, and

(d) subjecting the test and/or reference sample of step (a) to tandem mass spectrometry (MS-MS), in order to identify the differentially expressed peptides and/or the proteins from which they derive thus providing a candidate marker protein or marker peptide.

In this method, microdissected hypertrophied and normal blood vessels of the brain were used. The peptides of the enzymatically digested proteins derived from the small numbers of cells obtained by microdissection, were measured by MALDI-FT mass spectrometry. The identification of differentially expresses peptides was achieved by combining nano-LC fractionation of samples with offline MALDI-TOF/TOF and MALDI FTMS measurements. The findings were validated by using specific antibodies. Details of these experiments are described in the Examples below.

By using the above method, the inventors discovered a proteinaceous marker, colligin 2, the expression level of which was indicative for glioma.

Because gliomas are among neoplasms with highest degree of vascularisation and the growth of gliomas largely depends on angiogenetic processes, this finding indicates that colligin 2 can be used to detect vascularisation or angiogenetic processes associated with growth of tumors, ischemia or wound healing.

Suitable body fluid samples wherein the expression level of this marker is to be detected include blood, serum or cerebrospinal fluid samples. The body tissue sample wherein the expression level of the markers may be detected may be any body tissue, preferably however, the tissue is a blood vessel, still more preferably a blood vessel of the brain, most preferably a blood vessel of a suspect glioma or confirmed glioma.

The marker of the present invention is very suitably used in a method for monitoring the disease activity of tumors or the response of the patient to treatment regimens aimed at blood vessel or tissue repair after ischemia or other tissue or blood vessel trauma, or aimed at reducing tumor growth. Such a method comprises the step of measuring the expression level of colligin 2 in blood, CSF or vasculature of tumor tissue or healing tissue of wounds. Reference values for markers may be determined as described below and methods of diagnosis of glioma may be performed as described in the Examples below.

Generally, the marker is detected in amounts of around 0.1-100 femtomole per volume of 100-200 cells, preferably 0.5-5 fmole/100-200 cells, and generally around about 1 fmole/100-200 cells. The skilled person will understand that the exact value will depend on the tissue and on the normal values (reference values) measured in normal, healthy tissue. The skilled artisan is well aware of methods to obtain reference values for diagnostic markers. Generally, typical reference samples will be obtained from subjects that are clinically well documented and that are free from the disease, if for instance a tumor is to be diagnosed. In such samples, normal (reference) concentrations of the marker proteins can be determined, for instance by providing the average concentration over the reference population. In determining the reference concentration of the marker concentration a variety of considerations is taken into regard. Among such considerations are the type of disease to be diagnosed, the location of disease and the type of sample involved (e. g., tissue or CSF), the patient's age, weight, sex, general physical condition and the like. For instance, a group of at least 2 to preferably more than 3 subjects, preferably ranked according to the above considerations, for instance from various age categories, are taken as reference group.

The marker of the present invention is absent in samples wherein no physiological or pathological blood vessel formation is present. In contrast, the marker is present in samples wherein physiological or pathological processes of blood vessel formation occur. For in stance in glioma vasculature, the colligin 2 protein can easily be detected by histochemical techniques, whereas in healthy tissue of the same subject, said marker cannot be detected.

In general, a level in the concentration of the marker that is increased at least 1.5-10 times, preferably 2-5 times, but suitably about 3 times, relative to concentration of the reference value is indicative of the presence of physiological or pathological blood vessel formation.

Depending on the normal (healthy) status, a marker indicative of physiological or pathological blood vessel formation as defined herein may be present in the diseased condition vs. absent in the normal condition. More often however, the level of expression of the marker will be altered, usually enhanced, so that elevated levels of the marker indicate the presence of the angiogenetic or vasculogenetic process, the presence of the disease or even the severity of the disease condition. Therefore, in some instances, quantitative detection of the colligin 2 marker and comparison with reference values is necessary in order to draw conclusions. The steps which must be taken in order for a diagnosis to be made are generally:
i) an examination phase involving the collection of data,
ii) a comparison of these data with standard values,
iii) a finding of any significant deviation during the comparison, and
iv) the attribution of the deviation to a particular clinical picture, i.e. the deductive medical or veterinary decision phase.

In methods of the present invention, step iv is generally excluded. The methods of the present invention in particular relate to the technical steps of providing samples and providing clinical data on marker concentrations, which steps proceed the deductive medical or veterinary decision phase.

Detection of the marker in a patient sample may be performed by any method available to the artisan. Generally, in order to detect the subtle concentration differences in the expression level of the marker, sophisticated methods are required. The skilled person is well acquainted with the various methods available, and these need not be described in great detail here.

In short, suitable methods include mass spectrometric methods such as those described and used herein, in particular in the Examples, and immunological detection methods.

Immunological detection methods (i.e. immunoassays) for determining the (quantitative) presence of a peptide or protein in a sample are well known to those of skill in the art. The markers identified by methods of the present invention can be employed as immunogens for constructing antibodies immunoreactive to a protein of the present invention for such exemplary utilities as immunoassays or protein purification techniques.

In another aspect, the present invention provides for the use of a disease marker, identified by a method for identifying a disease marker according to the invention, in diagnosis, prognosis, or therapeutic monitoring of physiological or pathological blood vessel formation.

Polyclonal and monoclonal antibodies raised against colligin 2 protein or peptide fragments thereof and that bind specifically thereto can be used for detection purpose in the present invention, for example, in immunoassays in which they can be utilized in liquid phase or bound to a solid phase carrier. In addition, the monoclonal antibodies in these immunoassays can be detectably labeled in various ways. A variety of immunoassay formats may be used to select antibodies specifically reactive with a particular peptide or protein marker. For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective binding. Examples of types of immunoassays that can utilize monoclonal antibodies of the invention are competitive and non-competitive immunoassays in either a direct or indirect format. Examples of such immunoassays are the radioimmunoassay (RIA) and the sandwich (immunometric) assay.

Detection of the peptide or protein marker using an antibody can be done utilizing immunoassays that are run in either the forward, reverse, or simultaneous modes, including immunohistochemical assays on physiological samples. Those of skill in the art will know, or can readily discern, other immunoassay formats without undue experimentation.

Immunohistochemical staining may for instance be performed following the manufacturer's procedure (alkaline phosphatase technique) using a mouse monoclonal antibody for colligin 2 at a 1:500 dilution (Stressgene, Victoria, British Columbia, Canada). Paraffin sections (for instance having a thickness of 5 μm) may be mounted onto microslides that are for instance poly(L-lysine)-coated. Thereafter, the paraffin sections may be deparaffinized in xylene for 15 min, rehydrated through graded alcohol series, and then washed with water. The sections can then be washed with PBS and incubated with the antibody for a duration of for instance 30 min. After washing away the unreacted antibody with PBS, the detection reagent (for instance a secondary antibody with alkaline phosphatise enzyme) can be added and following an incubated for, for instance, 30 min at room temperature, the alkaline phosphatase substrate solution can be added to the sections which are then again incubated for about 30 min. Thereafter the sections can be washed with tap water, counterstained, and coverslipped with permanent mounting medium.

Antibodies can be bound to many different carriers and used to detect the presence of the disease markers. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

The binding of the antibody to the marker of the present invention can be detected in numerous ways that are well known in the art. Binding of the antibody and disease marker forms an immune complex that can be detected directly or indirectly. The immune complexes are detected directly, for example, when the antibodies employed are conjugated to a label. The immune complex is detected indirectly by examining for the effect of immune complex formation in an assay medium on a signal producing system or by employing a labeled receptor that specifically binds to an antibody of the invention. Suitable detection techniques that may be applied in concert with the above techniques include autoradiographic detection techniques, detection techniques based on fluorescence, luminescence or phosphorescence or chromogenic detection techniques. These detection techniques are known in the art of detection of biomolecules.

Use may for instance be made of signal producing systems, involving one or more components, at least one component being a detectable label, which generate a detectable signal that relates to the amount of bound and/or unbound label, i.e. the amount of label bound or not bound to the compound being detected. The label is any molecule that produces or can be induced to produce a signal, and preferably is a fluorescer, radio-label, enzyme, chemiluminescer or photosensitizer. Thus, the signal is detected and/or measured by detecting fluorescence or luminescence, radioactivity, enzyme activity or light absorbance.

Suitable labels include, by way of illustration and not limitation, enzymes such as alkaline phosphatase, glucose-6-phosphate dehydrogenase (“G6PDH”) and horseradish peroxidase; ribozyme; a substrate for a replicase such as QB replicase; promoters; dyes; fluorescers, such as fluorescein, rhodamine compounds, phycoerythrin, phycocyanin, allophycocyanin, o-phthaldehyde, and fluorescamine; chemiluminescers such as isoluminol; sensitizers; coenzymes; enzyme substrates; radiolabels such as ¹²⁵I, ¹⁴O, ³H, ⁵⁷Co and ⁷⁵Se; particles such as latex or carbon particles; metal sol; crystallite; liposomes; cells, etc., which may be further labeled with a dye, catalyst or other detectable group. Suitable enzymes and coenzymes are disclosed in U.S. Pat. No. 4,275,149; U.S. Pat. No. 4,318,980; suitable fluorescers and chemiluminescers are disclosed i.a. in U.S. Pat. No. 4,275,149.

There are numerous methods by which the label can produce a signal detectable by external means, for example, desirably by visual examination or by electromagnetic radiation, heat, and chemical reagents. The label or other signal producing system component can also be bound to a specific binding partner, another molecule or to a support.

The label can directly produce a signal, and therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

Alternately, the label may need other components to produce a signal, and the signal producing system would then include all the components required to produce a measurable signal, which may include substrates, coenzymes, enhancers, additional enzymes, substances that react with enzymic products, catalysts, activators, cofactors, inhibitors, scavengers, metal ions, and a specific binding substance required for binding of signal generating substances. A detailed discussion of suitable signal producing systems can be found in U.S. Pat. No. 5,185,243.

The label can be bound covalently to numerous specific binding partners: an antibody; a receptor for an antibody; a receptor that is capable of binding to a small molecule conjugated to an antibody; or a ligand analog. Bonding of the label to the specific binding partner may be accomplished by chemical reactions which result in replacing a hydrogen atom of the label with a bond to the specific binding partner member or may include a linking group between the label and the specific binding partner. Other signal producing system components may also be bound covalently to specific binding partners. For example, two signal producing system components such as a fluorescer and quencher can each be bound to a different antibody that forms a specific complex with the analyte.

Formation of the complex brings the fluorescer and quencher in close proximity, thus permitting the quencher to interact with the fluorescer to produce a signal. Methods of conjugation are well known in the art. See for example, U.S. Pat. No. 3,817,837. This invention also contemplates having an antibody bound to a first signal producing system component and a detectable label as the second signal producing system components. For example, when the detectable label is bound to a ligand analog, the extent of binding of the antibody to the analog can be measured by detecting the signal produced by the interaction of the signal producing system components.

Methods and means provided herein are particularly useful in a diagnostic kit for diagnosing a disease by immunological techniques. Such kits or assays may for example comprise one or more reference markers, one or more reference samples and/or one or more antibodies for any of the markers for the various disease conditions as described herein, and can be used specifically to carry put a method or use according to the present invention,

Methods for measuring the expression level of peptides or proteins by MALDI techniques as referred to herein are well known in the art and specific reference is made to the Experimental part described herein below.

The invention will now be illustrated by the following non-limiting Examples.

### Examples

### Example 1

### Identification of Glioma Neovascularization-Related Proteins by Using MALDI-FTMS and Nano-LC Fractionation to Microdissected Tumor Vessels

### 1.1. Material and Methods

### 1.1.1. Sampling

Ten fresh-frozen samples of glioblastoma located in the cerebral hemispheres and 10 samples of normal control hemispheric brain were taken form the files of the Department of Pathology, Erasmus M C, Rotterdam (Table 1).

TABLE 1
Clinical data

| Sample ID. | Sex | Age | |
|---|---|---|---|
| | | | Tumor location |
| G1 | m | 57 | Ri F |
| G2 | m | 57 | Le T |
| G3 | m | 55 | Ri F |
| G4 | m | 51 | Ri F |
| G5 | m | 51 | Le T |
| G6 | m | 48 | Le F |
| G7 | m | 47 | Ri O |
| G8 | m | 36 | Le P |
| G9 | m | 32 | Bi F |
| G10 | f | 30 | Ri F |
| | | | Cause of death |
| N1 | f | 76 | pneumonia |
| N2 | f | 62 | Cirrhosis + hepatocellular carcinoma |
| N3 | m | 62 | Ischemic cardiac disease |
| N4 | f | 60 | nasopharyngeal carcinoma |
| N5 | m | 48 | SAB/aneurysm |
| N6 | f | 48 | SAB/aneurysm |
| N7 | f | 39 | SAB/aneurysm |
| N8 | m | 34 | Brain stem abscess |
| N9 | m | 28 | hypertensive stroke |
| N10 | m | 24 weeks | intra-uterine infection |

G samples indicate Glioma patients
N samples indicate control patients

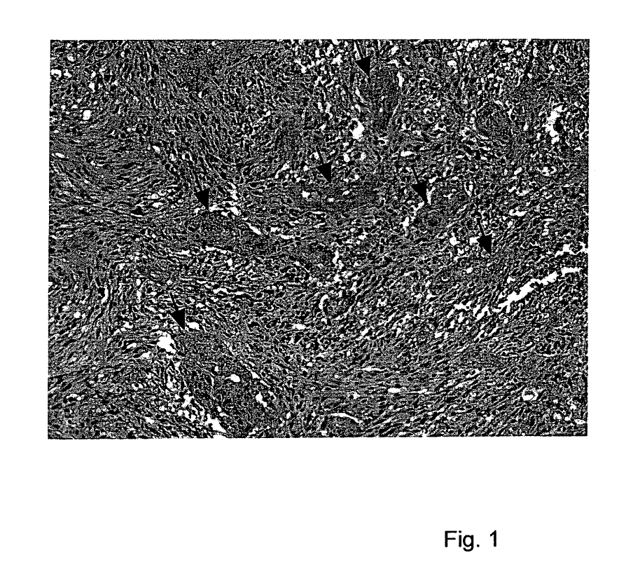

Sections of 5 mm from each sample were made, counterstained and examined by the neuropathologist (JMK) to verify the presence of proliferated tumor vessels (FIG. 1). The control samples of normal brains were subjected to the same procedure for the identification of the blood vessels.

### 1.1.2. Laser Capture Microdissection

Cryosections of 8 mm were made from each sample and mounted on polyethylene naphthalate (PEN) covered glass slides (P.A.L.M. Microlaser Technologies AG, Bernried, Germany) as described previously [Umar, A., et al., Proteomics, 2005. 5(10): p. 2680-8]. The slides were fixed in 70% ethanol and stored at −20° C. for not more than 2 days. After fixation and immediately before microdissection, the slides were washed twice with Milli-Q water, stained for 10 seconds in haematoxylin, washed again twice with Milli-Q water and subsequently dehydrated in a series of 50, 70, 95 and 100% ethanol solution and air dried. The P.A.L.M. laser microdissection and pressure catapulting (LMPC) device, type P-MB was used with PalmRobo v2.2 software at 40× magnification. Estimating that a cell has a volume of 10×10×10 mm, we microdissected an area of about 190,000 mm2 of blood vessels and another area of the same size of the surrounding tumor tissue from each sample, resulting in approximately 1,500 cells per sample. A total of 40 samples were collected, viz., 10 glioma vessels, 10 fields of glioma tissue surrounding the glioma vessels, 10 normal vessels and 10 fields of normal tissue surrounding the normal vessels. As a negative control, a corresponding area of the PEN membrane only was microdissected and analysed in the same way as the other samples. This negative control experiment was performed in three folds.

The microdissected cells were collected in the caps of P.A.L.M. tubes in 5 ml of 0.1% RapiGest buffer (Waters, Milford, Mass., USA). The caps were cut and placed onto 0.5 ml Eppendorf protein LoBind tubes (Eppendorf, Hamburg, Germany). Subsequently, these tubes were centrifuged at 12,000 g for 5 minutes. To make sure that all the cells were covered with buffer, another 5 ml of RapiGest was added to the cells. After microdissection, all samples were stored at −80° C.

### 1.1.3. Sample Preparation

After thawing the samples, the cells were disrupted by external sonification for 1 minute at 70% amplitude at a maximum temperature of 25° C. (Bransons Ultrasonics, Danbury, USA). The samples were incubated at 37° C. and 100° C. for 5 and 15 minutes respectively, for protein solubilization and denaturation. To each sample, 1.5 ml of 100 ng/ml gold grade trypsin (Promega, Madison, Wis., USA) in 3 mM Tris-HCL diluted 1:10 in 50 mM NH4HCO3 was added and incubated overnight at 37° C. for protein digestion. To inactivate trypsin and to degrade the RapiGest, 2 ml of 500 mM HCL was added and incubated for 30 minutes at 37° C. Samples were dried in a speedvac (Thermo Savant, Holbrook, N.Y., USA) and reconstituted in 5 ml of 50% acetonitrile (ACN)/0.5% trifluoroacetic acid (TFA)/water prior to measurement. Samples were used for immediate measurements, or stored for a maximum of 10 days at 4° C.

### 1.1.4. MALDI-FTMS Measurements and Data Analysis

### 1.1.4.1. MALDI-FTMS Measurements

Samples were spotted onto a 600/384 anchorchip target plate (Bruker Daltonics, Leipzig, Germany) in duplicate. Half a microliter of each sample was mixed on the spot with 1 ml of a 2,5-dihydroxybenzoic acid (DHB) matrix solution (10 mg/mL in 0.1% TFA)/water and the mixture was allowed to dry at ambient temperature. The MALDI-FTMS measurements were performed on a Bruker Apex Q instrument with a 9.4 T magnet (Bruker Daltonics, Bremen, Germany). For each measurement, 450 scans of 10 shots each were accumulated with 60% laser power. Mass spectra were acquired in the mass range of 800 to 4,000 Da. FTMS spectra were processed with a Gaussian filter and 2 zero fillings.

### 1.1.4.2. MALDI-FTMS External and Internal Calibration

A standard peptide calibration mix (Bruker Daltonics, Leipzig, Germany) which contains angiotensin I and II, substance P, Bombesin, Renin Substrate, ACTH clip 1-17, ACTH clip 18-39 and Somatostatin 28 was used for external calibration. To obtain better mass accuracies, an additional post-acquisition internal calibration step in DataAnalysis v3.4, built 169 software (Bruker Daltonics, USA) was performed. Ubiquitous actin peptide masses (m/z 1198.70545, 1515.74913, 1790.89186, 2215.06990 and 3183.61423) were used for internal calibration. To assess the accuracy of the measured masses, the peptides derived from keratin [Q8N175] present in the samples were compared to the calculated masses (1165.58475, 1234.67896, 1365.63930, 1381.64814, 1390.68085, 1707.77211, 1797.01161 and 2096.04673).

### 1.1.4.3. Data Analysis

Mono-isotopic peaks with S/N>3 were annotated with the SNAP algorithm using the pre-release version of DataAnalysis software package (v3.4, built169). The peak lists were saved in a general text format, which was used as an input for a home made script in the R-program, (www.r-project.org). With this script a matrix file was generated, indicating the presence or absence of each peptide mass in the different mass spectra. If a specific peptide appeared at least in 5 samples for each group and never appeared in the other groups, it was considered as a group specific peptide. In this way, a list of differentially expressed peptides was generated. These masses of the differentially expressed peptides were submitted to the MASCOT search engine (Matrix Science, London, UK) using the SWISS-PROT (40.21) database, allowing 1 ppm peptide mass tolerance and one missed trypsin cleavage site. In addition, we performed Hierarchial Clustering based on masses and the group of samples using the matrix file in the Spotfire software (Spotfire, Somerville, Mass., USA).

### 1.1.5. Sample Preparation for Nano-LC

Sample G8 was selected for fractionation (Table 1). One, 4 and 8 frozen sections were made, respectively. These sections from the entire tumor sample including the vessels were prepared as described above. Each section contained about 2,000,000 cells of which an estimated 10% were blood vessel derived cells. Twenty ml RapiGest buffer was added (Waters, Milford, Mass., USA) to the frozen sections followed by 1 minute sonification, 5 minutes at 37° C. and finally 15 minutes at 100° C. For each section 1 ml of 100 ng/ml gold grade trypsin (Promega, Madison, Wis., USA) in 3 mM Tris-HCL was added and samples were incubated overnight at 37° C. Finally, 50 mM HCL was added. For comparison, 8 sections from normal brain sample N5 were prepared in exactly the same way.

In addition, an area of about 900,000 mm2 of blood vessels from each of the glioma samples and the normal control samples were microdissected and pooled, resulting in one sample of glioma blood vessels and one sample consisting of control blood vessels. Pooling of the samples was necessary because the nano-LC procedure requires far more tissue than obtained by microdissection. Twenty ml RapiGest buffer was added and the samples were stored at about 80° C. All samples were subjected to the nano-LC fractionation immediately after preparation.

### 1.1.6. Fractionation by Nano-LC

Fractionation was performed using a C18 Pep Map column (75 mm i.d.×150 mm, 3 mm, Dionex, Sunnyvale, Calif., USA). Five ml of the sample was loaded onto the trap column (300 mm i.d.×5 mm, 5 mm, Dionex, Sunnyvale, Calif., USA). Fractionation was performed for 130 minutes with a gradient of buffer A (100% H2O, 0.05% TFA) and buffer B (80% ACN, 20% H2O and 0.04% TFA); 0 to 15 min, 0% buffer B, 15.1 min 15%, 75 min 40%, 90 min 70%, 90.1-100 min 95%, 100.1 min 0% and 130 min 0%. Fifteen second fractions of the sample were spotted automatically onto 384 prespotted anchorchip plates (Bruker Daltonics, USA) containing a-cyano-4-hydroxycinnamic acid (HCCA) matrix, using a robotic system (Probot Micro Fraction Collector, Dionex, Sunnyvale, Calif., USA). To each fraction, 1 ml water was added. Finally, we used a 10 mM (NH)₄H₂PO₄ in 0.1% TFA/water solution to wash the pre-spotted plate for 5 seconds to remove salts. The plates were subsequently measured by automated MALDI-TOF/TOF (Ultraflex, Bruker Daltonics, Germany) using WARLP-LC software. MS spectra of each individual spot were obtained. Spots and peptide masses for performing MS/MS measurements were determined automatically by the WARLP-LC software. A file containing the MS and the MS/MS peak lists was submitted to the MASCOT search engine (Matrix Science, London, UK) using the SWISS-PROT (40.21) database allowing 150 ppm parent mass tolerance, 0.5 Dalton fragments tolerance and one missed trypsin cleavage site. In addition, identification was confirmed by exact mass measurements on the MALDI-FTMS, adding 1 mL DHB solution to the fractionated spot and allowed to dry:

### 1.1.7. Backward Database Searching

By in silico digestion of the identified proteins, theoretical peptides were generated which were sought for in the monoisotopic peaks of the MALDI-FTMS.

The (UniProtKB/Swiss-Prot) accession number for all of the identified proteins was entered into the peptide cutter program (www.expasy.org/tools/peptidecutter), choosing trypsin as enzyme for digestion and allowing one trypsin missed cleavage site. All the possible tryptic fragments from each protein were compared with the peptide masses obtained by MALDI-FTMS within 0.5 ppm (the internal calibration). The distribution of the matched peptides over the four groups was checked manually.

### 1.1.8. Immunohistochemical Staining

The expression of fibronectin and colligin 2 in glioma blood vessels was confirmed by immunohistochemistry using specific antibodies against these proteins on paraffin sections of the samples. We first confirmed our results using the 10 glioma samples and the 10 normal brain samples that were used in our proteomics approach. To investigate the expression variation between the two groups, an additional six samples of glioma and four samples of normal brain were examined. In addition, a series of other gliomas, carcinomas, vascular malformations, other reactive conditions in which neoangiogenesis takes place, and tissues with notorious neoangiogenesis were also tested for the presence of these proteins (Table 2).

TABLE 2
Samples used for immunohistochemistry (IR, immunoreactivity).

| | | IR for | IR for |
|---|---|---|---|
| | No. of | colligin 2 in | fibronectin in |
| Sample type | samples | blood vessels | blood vessels |
| Normal brain samples | 14^{a} | Negative | Negative/faint |
| Glioma | | | |
| Glioblastoma | 16^{b} | Positive | Positive |
| Pilocytic astrocytoma | 3 | Positive | Positive |
| Ependymoma | 3 | Positive | Positive |
| Myxopapillary ependymoma | 2 | Positive | Positive |
| Anaplastic oligodendroglioma | 6 | Positive | Positive |
| Renal cell carcinoma | 5 | Positive | Positive |
| Vascular malformation | | | |
| AVM | 5 | Negative | Positive |
| Cavernous hemangioma | 2 | Negative | Positive |
| Reactive condition | | | |
| Subdural membrane | 2 | Positive | Positive |
| Contusio cerebri | 2 | Positive | Positive |
| Ischemic infarction of brain | 2 | Positive | Positive |
| Inflammation (outside brain) | 5 | Positive | Positive |
| Tissues with notorious | | | |
| neoangiogenesis | | | |
| Placenta | 6 | Positive | Positive |
| Endometrium | 6 | Positive | Positive |

^{a}10 samples used for MALDI-FTMS plus an additional four samples.
^{b}10 samples used for MALDI-FTMS plus an additional six samples.

Immunohistochemical staining was performed following the manufacturer's procedure (alkaline phosphatase technique) using rabbit polyclonal antibody for fibronectin at a 1:1000 dilution (DakoCytomation, Glostrup, Denmark) and mouse monoclonal antibody for colligin 2 at a 1:500 dilution (Stressgene, Victoria, British Columbia, Canada). Paraffin sections (5 μm) were mounted onto poly(L-lysine)-coated microslides, deparaffinized in xylene for 15 min, rehydrated through graded alcohol, and then washed with water. The sections were washed with PBS and incubated with the antibody for 30 min. After washing the sections with PBS, the corresponding antigen was added and incubated for 30 min at room temperature. New fuchsin alkaline phosphatase substrate solution was freshly prepared, and the sections were incubated for about 30 min. Afterward the sections were washed with tap water, counterstained, and coverslipped with permanent mounting medium.

### 1.2. Results

### 1.2.1 FTMS Measurements

The MALDI-FTMS measurements of the microdissected samples yielded approximately 700-1,100 monoisotopic peaks for almost all spectra. Only one glioma vessel and one normal tissue sample contained less than 100 peaks. However, these spectra were not excluded from our analysis. An accuracy of 3 ppm was obtained by external calibration using a standard peptide calibration mix. After internal calibration the accuracy increased below 0.5 ppm.

### 1.2.2. FTMS Data Analysis

Following our strict criteria, a list of 16 differentially expressed peptides was obtained (Table 3). All 16 peptides were expressed in the glioma vessel group only. The MASCOT database search resulted in matching of four out of the 16 peptides to fibronectin precursor protein [P02751]. In order to exclude that matching of the four peptides to fibronectin was just by chance, the following database searches were performed. We added the integers 10, 11, 12, until 30 Daltons to the masses of the 16 peptides which were found for 20 additional searches. By this procedure no proteins were found to match by chance with four peptides. At maximum, only one peptide matched to one protein in the MASCOT database. This virtually ruled out the possibility of randomly finding fibronectin.

TABLE 3
List of differentially expressed peptides

| | Number of samples in which these peptides were found: | | | |
|---|---|---|---|---|
| Peptides | | Glioma | | Normal brain |
| (measured | | surrounding | Normal brain | surrounding |
| masses) | Glioma vessels | tissue | vessels | tissue |
| 1926.04620* | 8 | 0 | 0 | 0 |
| 2470.32072* | 6 | 0 | 0 | 0 |
| 1593.81172* | 5 | 0 | 0 | 0 |
| 1807.90584* | 5 | 0 | 0 | 0 |
| 1535.72354* | 5 | 0 | 0 | 0 |
| 2257.07971* | 5 | 0 | 0 | 0 |
| 1659.80041* | 5 | 0 | 0 | 0 |
| 1275.55961* | 5 | 0 | 0 | 0 |
| 1731.89535* | 5 | 0 | 0 | 0 |
| 1116.54323 | 5 | 0 | 0 | 0 |
| 1849.85488 | 5 | 0 | 0 | 0 |
| 2089.00769 | 5 | 0 | 0 | 0 |
| 2157.10653 | 5 | 0 | 0 | 0 |
| 2164.00992 | 5 | 0 | 0 | 0 |
| 2530.25829 | 5 | 0 | 0 | 0 |
| 2642.21770 | 5 | 0 | 0 | 0 |

*Peptides resulted in protein identification.

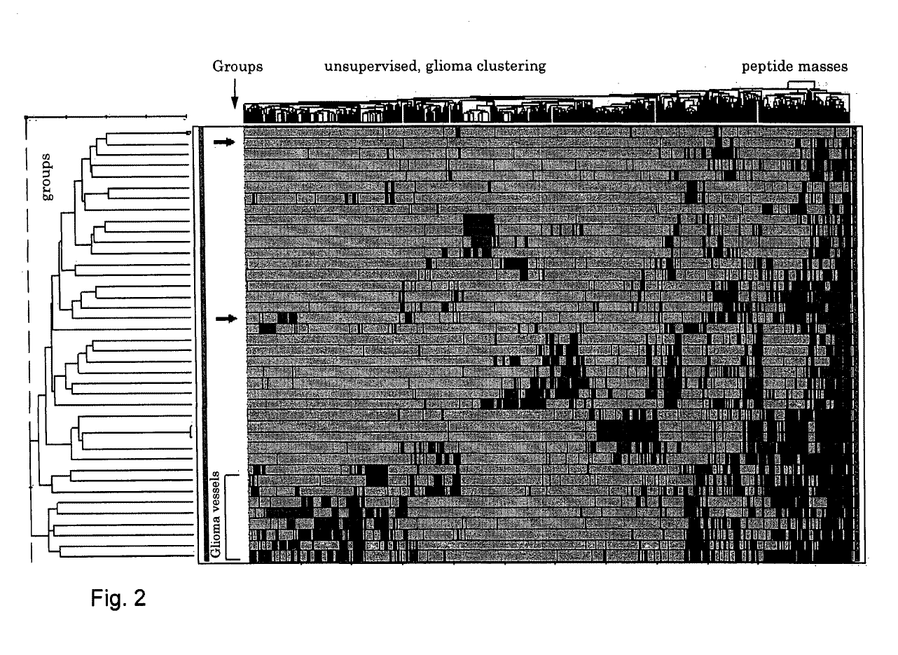

FIG. 2 shows the result of the unsupervised cluster analysis in two directions; peptide masses and groups of samples in the Spotfire program. A cluster of eight glioma vessel samples is observed. From the two samples which did not cluster, one had a poor spectrum (<100 peaks); this sample clustered with the sample from normal tissue at the top of the heat map which also displayed a poor spectrum. The other one did not cluster with any group. Within the peptide masses, a specific pattern of glioma blood vessels is recognized.

### 1.2.3. Nano-LC Fractionation/MALDI-TOF-MS/MS

Pooling small number of cells collected by microdissection before nano-LC fractionation resulted in the identification of some high abundant proteins, among which fibronectin. To identify more proteins, we increased the number of cells by using whole sections of glioma and normal samples. The number of identified peptides was increased and the maximum was reached with the injection of eight sections (Table 4). The capacity of the nanoLC column did not allow further expansion of the number of sections. Fractionation of eight sections led to the significant identification of 189 proteins, with a minimum mowse score of 24 for MS/MS.

TABLE 4
Results for the various numbers of sections used
for fractionation in the nano-LC:

| | | | | Normal | Glioma | Normal brain |
|---|---|---|---|---|---|---|
| | Glioma | Glioma 4 | Glioma 8 | brain | microdiss. | microdiss. |
| Sample type | 1 section | sections | sections | 8 sections | cells^{a} | cells^{a} |
| No. of MS | 2307 | 3328 | 3383 | 2985 | 552 | 779 |
| measurements | | | | | | |
| No. of MS/MS | 734 | 1194 | 2160 | 1752 | 368 | 416 |
| measurements | | | | | | |
| No. of | 32 | 131 | 189 | 140 | 27 | 13 |
| identified | | | | | | |
| proteins | | | | | | |

^{a}15,000 microdissected cells

The data obtained from MALDI-TOF/TOF after the fractionation procedure were compared to the MALDI-FTMS data, searching specifically for the 16 differentially expressed peptides. Nine out of 16 peptides matched within 200 ppm. To obtain a higher mass accuracy for the peptides, the corresponding spots of these nine peptides were re-measured in the MALDI-FTMS. The exact mass of five out of nine peptides matched within 3 ppm (external calibration) with the masses originally obtained by FTMS. In order to relate these peptides to proteins, the MS/MS data of these peptides were searched for in the database, resulting in a significant matching of four of them (sequence score >24). Two peptides matched to fibrinogen beta chain precursor [p02675], one peptide to colligin 2 [P50454] and one peptide to acidic calponin 3 [Q15417]. In the MALDI-TOF data set more peptides belonging to these proteins were sought and an additional three peptides belonging to fibrinogen beta chain precursor, and two belonging to colligin 2 protein, were found. We also found an additional 17 peptides from fibronectin, of which nine had a significant MS/MS score.

### 1.2.4. Backward Database Searching

The search of the peak list obtained from the In silico digestion of fibronectin sequence in the FTMS data resulted in the finding of six extra peptides. Five peptides were found in the glioma vessels group only, and one was also seen in one sample of the normal brain blood vessels (Table 5). The same search for the in silico digestion of fibrinogen yielded nine additional peptides of which three were exclusively found in the glioma vessels group and the others in one sample of the normal vessels (Table 6). Searching for the theoretical peptides of colligin 2 and acidic calponin3 did not result in the finding of any extra peptide.

TABLE 5
Differentially expressed Fibronectin precursor [P02751] peptides.

| | | | Number of samples in which | | | |
|---|---|---|---|---|---|---|
| | | | these peptides were found: | | | |
| | Exact | | | | | Normal |
| Fibronectin | fibronectin | | | Glioma | Normal | brain |
| peptides found | peptide | | Glioma | surrounding | brain | surrounding |
| in FTMS spectra | masses | Δ ppm | vessels | tissue | vessels | tissue |
| 1926.04620^{a} | 1926.04833 | 1.11 | 8 | 0 | 0 | 0 |
| 2470.32072^{a} | 2470.31874 | 0.80 | 6 | 0 | 0 | 0 |
| 1593.81172^{a} | 1593.81188 | 0.05 | 5 | 0 | 0 | 0 |
| 1807.90584^{a} | 1807.90471 | 0.63 | 5 | 0 | 0 | 0 |
| 1629.87232^{b} | 1629.87070 | 0.99 | 4 | 1 | 0 | 0 |
| 2692.37550^{b} | 2692.37292 | 0.97 | 4 | 0 | 0 | 0 |
| 1349.68509^{b} | 1349.68481 | 0.21 | 3 | 0 | 0 | 0 |
| 1401.66582^{b} | 1401.66582 | 0.01 | 3 | 0 | 0 | 0 |
| 2524.36562^{b} | 2524.36567 | 0.03 | 3 | 0 | 0 | 0 |
| 3042.59234^{b} | 3042.58942 | 0.96 | 3 | 0 | 0 | 0 |

^{a}Peptides matching the criteria used in this Example.
^{b}Peptides derived from in silico digestion.

TABLE 6
Peptides derived from in silico digestion of fibrinogen

| | Exact | | | | | |
|---|---|---|---|---|---|---|
| Fibrinogen | fibrinogen | | Number of samples in which | | | |
| peptide | masses | | these peptides were present in: | | | |
| masses | derived | | | | | Normal |
| found in | from in | | | Glioma | Normal | brain |
| the FTMS | silico | | Glioma | surrounding | brain | surrounding |
| spectra | digestion | Δ ppm | vessels | tissue | vessels | tissue |
| 1032.56252 | 1032.5625 | 0.02 | 5 | 0 | 0 | 0 |
| 1239.51764 | 1239.5177 | 0.05 | 5 | 0 | 0 | 0 |
| 2385.17568 | 2385.1754 | 0.12 | 4 | 0 | 0 | 0 |
| 1275.55961 | 1275.5600 | 0.3 | 4 | 1 | 0 | 0 |
| 1544.69498 | 1544.6950 | 0.01 | 3 | 1 | 0 | 0 |
| 1668.71478 | 1668.7151 | 0.2 | 3 | 1 | 0 | 0 |
| 886.38736 | 886.3876 | 0.3 | 2 | 1 | 0 | 0 |
| 1951.00371 | 1951.0031 | 0.3 | 2 | 1 | 0 | 0 |

### 1.2.5. Immunohistochemistry:

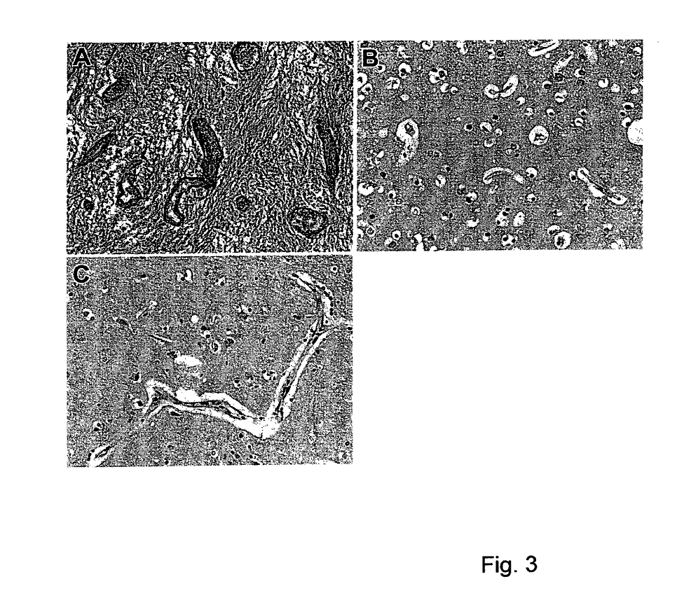

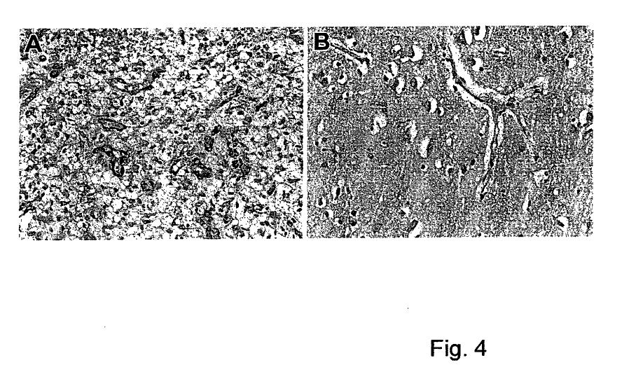

The expression of fibronectin and colligin 2 proteins in glioma blood vessels was confirmed by immunohistochemistry. The proliferated blood vessels present in glioblastoma samples were invariably immunopositive for fibronectin and colligin 2, whereas the blood vessels in the control brain samples remained negative (FIGS. 3 and 4). In a few capillaries of normal brain some fibronectin was expressed but to a far lesser extent as compared with the expression observed in the proliferated glioma vessels. The blood vessels in the arachnoidal space were immunopositive for fibronectin, not for colligin 2.

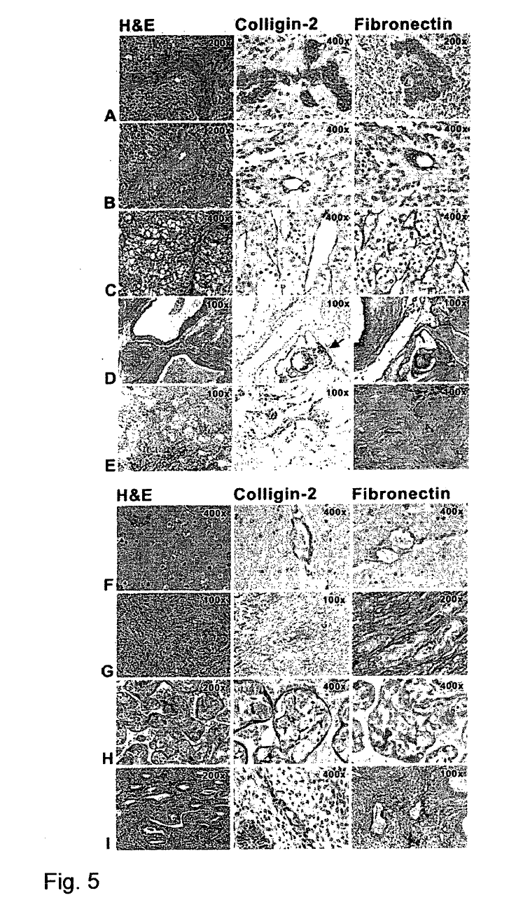

In FIG. 5 the results of additional immunostaining of various gliomas, carcinomas, vascular malformations, and tissues and reactive conditions in which neoangiogenesis takes place are shown. It appears that both colligin 2 and fibronectin are present in active angiogenesis in tumors, normal tissues, and reactive processes. For instance, the vascular malformations (arteriovenous malformation (AVM) and cavernous hemangioma) remained immunonegative for colligin 2, but in the arteriovenous malformation a spot of active angiogenesis, namely the recanalization of a vessel, was immunopositive (FIG. 5D).

### 1.3. Conclusion

In this Example it was attempted to identify angiogenesis-related proteins in glioma in the surgically removed specimens of patients suffering from glial tumors. To achieve this goal, relevant cell populations had to be targeted. Like all tissues, tumors consist of complex 3-dimensional structures of heterogeneous mixture of cell types. Laser microdissection provides an efficient and accurate method to obtain specific cell populations like glioma blood vessels in the present study. The hypertrophied vessel walls of glioma vasculature consist of endothelial cells, pericytes and cells expressing smooth muscle actin. In addition, these vessels may also contain glial tumor cells (mosaic vessels). In order to eliminate proteins derived from these tumor cells, we also microdissected glial tumor tissue for comparison. Any peptide present in the blood vessels that was also found in the glioma tissue was eliminated from the list of differentially expressed peptides. Therefore, comparison of the various microdissected tissues is essential for targeting structure-specific proteins.

Application of MALDI-FTMS holds significant advantages over that of other types of mass spectrometry. FTMS provides very high mass accuracies and its ability to perform an internal calibration increases the accuracy considerably. In the present study we achieved an accuracy of ±3 ppm by external calibration and up to ±0.5 ppm by internal calibration. One of the advantages of MALDI-FTMS is the very high mass resolution, which in the present study generated relatively complex spectra, consisted 700-1,100 mono isotopic peaks per spectrum. Yet, another advantage is the very high sensitivity of the FTMS, which is higher than any other mass spectrometric technique currently available. In addition, FTMS provides an excellent signal-to-noise ratio, since the source of noise in MALDI-FTMS is of physical origin and not a chemical based noise as generated in the MALDI-TOF. These advantages allow studying very small numbers of targeted cells.

The MALDI-FTMS measurements of microdissected samples enabled us to detect a specific peptide pattern for the distinct targeted cell populations, but the results were not adequate to directly identify all of their related proteins. The chance of identifying a protein based on accurate peptide masses rises by increasing the number of peptides generated and detected from that protein. The number detectable peptides per protein depend on some factors: the size of the protein, the chemical properties of both the protein and the derived peptides, the relative concentration of a protein and the enzyme used in digestion. Last but not least, protein identification by detection of peptides relies highly on the accuracy and completeness of available databases. In the present study we succeeded to identify the protein fibronectin based on the precise masses of four peptides generated by MALDI-FTMS.

The in silico digestion approach, appeared to be a valuable tool to confirm the presence of peptides derived from a specific proteins in the spectra obtained by MALDI-FTMS. The high peptide mass accuracy of MALDI-FTMS facilitates the match with the calculated masses generated by in silico digestion. Nevertheless, the nature of a protein, its concentration and its ionization ability still play major roles in the detection of peptides.

The complexity of the sample in combination with a relative low sensitive for MS/MS in FTMS on MALDI ions complicates the identification of peptides based on direct MS/MS measurements. To reduce those effects, we applied nano-LC fractionation prior to MALDI-TOF/TOF. Because the number of cells required for nano-LC fractionation is much higher than what is obtained from sample microdissection, we pooled the microdissected cells from all samples resulting in one sample of 15,000 cells. However the loss of sample during preparation steps and in the nano-LC column is still considerable. In addition the overall sensitivity of MALDI-TOF measurements is considerably less compared to MALDI-FTMS. These factors together resulted in the identification of only the high abundant proteins of the pooled microdissected cells. The identification of lower abundant proteins can be achieved by using more cells however the microdissecting approach is then riot longer feasible. The tryptic digest of whole sections allowed the identification of many more proteins in both glioma and normal brain samples, particularly when we used peptide concentrations close to the maximum capacity of the column (eight sections). Within the spectra that were generated by MALDI-TOF following nano-LC, we specifically sought the peaks that were previously identified by FTMS, i.e. the 16 differentially expressed peptides. The low percentage of vessels, which is at maximum 10% of the cells in a section, resulted in producing low number of peptides from their specific proteins. The detection of vessels specific peptides probably was masked by the detection of the high percentage peptides derived from the surrounding tissue. For that reason not all the 16 differentially expressed peptides found in the MALDI-FTMS experiments were observed after fractionation followed by MALDI-TOF/TOF. Yet, MS/MS data of four peptides were obtained and their identification was based on both, very accurate peptide masses and their significant MS/MS measurements. Importantly, fractionation also increased the number of peptides generated from a single protein, thus improving the confidence in the identification significantly (Table 7).

TABLE 7

Differentially expressed proteins identified by nano-LC fractionation

| Identified | | | | | | |
|---|---|---|---|---|---|---|
| protein | | | | | | Extra |
| (Accession | FTMS | Calculated | Δ ppm | Sequence | | peptides |
| no.) | mass¹ | mass | (score) | coverage² | Sequence³ | identified⁴ |
| Fibrinogen | 1535.72354 | 1535.72366 | 0.13 | 13% | AHYGGFTVQNEANK | 5 |
| βchain | | | (52) | | | |
| (P02675) | 2257.07971 | 2257.08046 | 0.35 | | GGETSEMYLIQPDSSVKPYR | |
| | | | (48) | | | |
| Colligin 2 | 1659.80041 | 1659.80126 | 0.54 | 6% | LYGPSSVSFADDFVR | 1 |
| (P50454) | | | (39) | | | |
| Acidic | 1275.55961 | 1275.56000 | 0.31 | 3% | YDHQAEEDLR | 0 |
| calponin | | | (39) | | | |
| (Q15417) | | | | | | |

¹Specific peptide masses by FTMS (pre-fractionation)
²Sequence coverage of protein
³Sequence obtained after nano-LC fractionation and MALDI-TOF/TOF measurements
⁴No. of extra peptides identified after nano-LC fractionation

Two of the four proteins identified by the proteomics approach were successfully validated by immunohistochemistry. The faint staining for fibronectin of some of the normal brain blood vessels is in line with the detection of one fibronectin peptide by mass spectrometry in the normal brain vessels (Table 5). The colligin 2 antibody appeared to be specific for the glioma vessels. The immunohistochemical validation of the findings by mass spectrometry highlights the sensitivity and accuracy of these techniques and illustrates its potential of identifying specific proteins. The additional immunostaining of various lesions and tissues demonstrates that colligin 2 and fibronectin both are expressed in the context of neoangiogenesis. The expression was not specific for glioma neovascularization but also was found in the proliferating blood vessels in other tumors. Moreover, it is also seen in non-neoplastic tissues in which angiogenesis takes place. Therefore, colligin 2 and fibronectin should be considered as participants in the process of neovascularization in general without specificity for tissue type.

So far, various growth factors taking part in the process of neoangiogenesis have been identified in gliomas, such as vascular endothelial growth factor and platelet-derived growth factor. Relations have been discovered between some cytokines such as transforming growth factor-β and tumor blood vessels. Furthermore, endogenous expression of angiogenesis inhibitor factors, e.g. angiostatin, endostatin, and thrombospondin-1 and -2, by glioma tumor vessels also have been reported. Some of these proteins have been used to monitor therapy effects. Despite the gradual unraveling of the roles of these regulatory proteins in the process of tumor neovascularization, no major steps forward in antiangiogenic therapies for gliomas have been recorded. The identification of more tumor vasculature-related proteins may increase the chance of finding targets for antiangiogenic therapies. Such discoveries may well increase our understanding of the formation of neovasculature in glioma.

In the present study, we identified fibronectin, fibrinogen, colligin 2, and acidic calponin 3 as proteins that are expressed in the glioma vasculature. Fibronectin is a high molecular weight, multifunctional matrix protein that binds to other extracellular matrix proteins such as collagen, fibrin, and heparin. Several studies addressed the relation between fibronectin and tumors, including breast cancer, melanoma and gliomas. Overexpression of fibronectin in glioblastoma as detected by immunohistochemistry was reported previously. The expression of fibronectin by glioma blood vessels suggests that this protein plays a role in the development of glioma vasculature. In a study using suppression subtractive hybridization in which pilocytic astrocytoma were compared with glioblastoma, fibronectin was found to be differentially expressed; the glioblastomas expressed fibronectin, whereas the pilocytic astrocytomas did not. However, we did not find a difference in the expression of fibronectin between these two tumor types. Because hypertrophied microvasculature is a hallmark of both glial tumor types, despite their different World Health Organization grades, this finding did not surprise us.

Colligin 2, also called heat shock protein-47, is a collagen-binding protein that is associated with an increase in the production of procollagen in human vascular smooth muscle cells. Colligin 2 has been related to angiogenesis in oral squamous cell carcinomas. Acidic calponin, also identified in this study, is a thin filament-associated protein detected in a number of different cells and tissues. It was mentioned among the differentially expressed proteins in human glioblastoma cell lines and tumors. Acidic calponin modulates the contraction of smooth muscle cells. Interestingly, the proteins found in the present study share their prominent role in cell motility. It may very well be that the identification of these proteins is a reflection of their up-regulation in glioma vasculature. During neoplastic angiogenesis, sprouting of pre-existent blood vessels stimulates motility of the activated endothelial cells involved in this process. Furthermore, the putative influx of angiogenic precursor cells from the bone marrow into glioma may require the activation of motility even more. Further studies may detail the function and interaction of the proteins found in this study.

1. A method for detecting physiological or pathological blood vessel formation in a subject, comprising determining the expression level of colligin 2 in blood, cerebrospinal fluid or tissue vasculature.

2. Method according to claim 1, wherein said physiological or pathological blood vessel formation is indicative of tumor activity.

3. Method according to claim 1, wherein said tissue is a tumor.

4. Method according to claim 1, wherein in addition to said expression level of colligin 2, also the expression level of one or more of the proteins selected from the group consisting of fibronectin, fibrinogen, and acidic calponin 3 is determined.

5. Method according to claim 1, wherein said expression level is determined by detecting the said protein or a peptide fragment thereof in a mass range of 800 to 27,000 Da.

6. Method according to claim 5, wherein said detection is performed by immunohistochemistry or mass spectrometry.

7. Use of a method according to claim 1, wherein said use is for monitoring:
a disease process;
a healing process; or
responsiveness to disease therapy.

8. Use according to claim 7, wherein said disease process is cancer or ischemia; or wherein said healing process is a wound healing process or tissue repair process following ischemia; or wherein said disease therapy is anti-tumor therapy.

9. Marker protein or marker peptide for detecting physiological or pathological blood vessel formation in a subject wherein said marker protein is colligin 2 and said marker peptide is a peptide fragment of colligin 2 having a mass of between 800 and 27,000 Da.

10. The marker protein or marker peptide according to claim 9, wherein said physiological or pathological blood vessel formation is related to vasculogenesis; ischemia; and/or wound healing.

11. Marker profile for detecting physiological or pathological blood vessel formation in a subject wherein said marker profile comprises the expression level in blood, cerebrospinal fluid or tissue vasculature of a subject of a first protein being colligin 2 or a peptide thereof, and wherein said marker profile further comprises at least one expression level of a protein or peptide fragment selected from the group of fibronectin, fibrinogen and acidic calponin 3.

12. Use of a marker protein or marker peptide for detecting physiological or pathological blood vessel formation in a subject, wherein the marker protein comprises colligin 2 and said marker peptide is a peptide fragment of colligin 2 having a mass of between 800 and 27,000 Da.

13. Use of a marker profile for detecting physiological or pathological blood vessel formation in a subject, wherein the marker profile comprises:
(a) the expression level in blood, cerebrospinal fluid, or tissue vasculature of a subject of a first protein being colligin 2 or a peptide thereof, and
(b) at least on expression level of a protein or peptide fragment selected from the group of fibronectin, fibrinogen and acidic calponin 3.

14. Method according to claim 1, wherein said physiological or pathological blood vessel formation is indicative of glioma activity, ischemia, and/or wound healing.

15. The method of claim 9, wherein said physiological or pathological blood vessel formation is related to tumorigenesis and/or glioma activity.
